(19) United States
(12) Patent Application Publication
(10) Pub. No.: US 2024/0191801 A1
(43) Pub. Date: Jun. 13, 2024

(54) VARIABLE ORIFICE ROTARY VALVE FOR CONTROLLING GAS FLOW

(71) Applicant: ORIGIN MEDICAL DEVICES INC., Newport Beach, CA (US)

(72) Inventors: Yuval GUR, Ra'anana (IL); Ory GUR, Ra'anana (IL)

(21) Appl. No.: 18/287,345

(22) PCT Filed: May 18, 2022
(86) PCT No.: PCT/IL2022/050519

§ 371 Date: Oct. 18, 2023

Related U.S. Application Data
(60) Provisional application No. 63/190,475, filed on May 19, 2021.

(51) Int. Cl. F16K 3/26; A61M 16/20; F16K 3/32; F16K 3/34; F16K 27/04

(52) U.S. Cl. CPC F16K 3/26; F16K 3/32; F16K 3/34; F16K 27/041; A61M 16/203; A61M 2205/103; F16K 2200/204

(57) ABSTRACT

A variable orifice rotary valve for controlling gas flow has a housing with a cylindrical interior passage, a housing opening extending from the interior passage through the housing and a rotatable valve element including a sidewall having a cylindrical external sidewall surface. A slot in the sidewall varies in depth linearly or nonlinearly along at least a portion of a circumferential length of the slot and terminates in an opening in the sidewall. The valve element is rotatably received within the interior passage such that rotation of the valve element determines a length and depth of the portion of the slot that overlaps with the housing opening. That portion of the slot comprises part of the fluid channel. A gas flow rate through the fluid channel is determined by the width, length and depth of the portion of the slot that overlaps with the housing opening.

### TECHNICAL FIELD

This disclosure generally relates to variable orifice valves that are configured and operable to control the flow of gases.

### BACKGROUND

Potential drawbacks to known valves include limited ability to i) provide fine control over the flow restriction; ii) provide relatively linear control of the flow; iii) achieve fast response time from fully closed to fully open without excessive power consumption; iv) be easily controlled electronically; v) control flow over a very wide range of flow rates; vi) function without lubrication; vii) in ventilation applications, for example, involving blowers, enable control of flow of air or oxygen or a gas mixture to the patient, and viii) control flows at very low pressures, such as in ventilation applications, due to the size of the orifice opening required.

There is thus a need in the art for improved valves configured to provide flow of gases in a controlled manner.

### SUMMARY OF THE EMBODIMENTS

One embodiment is a variable orifice rotary valve for controlling gas flow, comprising a housing of the valve including a substantially cylindrical interior passage, and a housing opening extending from the interior passage through the housing; a rotatable valve element comprising a sidewall having a cylindrical external sidewall surface and an internal wall surface, the valve element having a main opening, a slot in the sidewall, the slot varying in depth along a circumferential length of the slot and terminating in an opening in the sidewall, the valve element rotatably received within the interior passage of the housing such that a rotation of the valve element within the interior passage determines a circumferential length and a depth of a portion of the slot that overlaps with the housing opening.

In some embodiments, at a position of the valve element in which the slot overlaps with the housing opening, the slot comprises part of a fluid channel from the main opening of the valve element to and through the housing opening, a gas flow rate through the fluid channel determined at least in part by the circumferential length and the depth of the portion of the slot that overlaps with the housing opening.

In some embodiments, a rate of gas flow varies with a rotational angle of the valve element.

In some embodiments, a profile of the slot is tongue-like.

In some embodiments, the depth of the slot varies substantially along a direction of rotation of the valve element.

In some embodiments, the depth of the slot varies linearly along the circumferential length of the slot. In some embodiments, the depth of the slot varies gradually.

In some embodiments, the depth of the slot varies nonlinearly along the circumferential length of the slot. In some embodiments, the depth of the slot varies gradually.

In some embodiments, a rate of change of the depth of the slot varies linearly through at least a portion of the slot.

In some embodiments, a rate of change of the depth of the slot varies non-linearly through at least a portion of the slot

In some embodiments, the depth of the slot varies through a first portion of the slot at a first steepness and then varies through a second portion of the slot at a second steepness.

In some embodiments, the depth of the slot increases through a first portion of the slot and decreases through a second portion of the slot.

In some embodiments, the depth of the slot varies nonlinearly through a first portion of the slot and varies linearly through a second portion of the slot.

In some embodiments, the depth of the slot varies through a first portion of the slot and does not vary through a second portion of the slot, the second portion bounded by the slot floor surface.

In some embodiments, in at least a portion of the slot a width of the slot varies as the depth of the slot varies.

In some embodiments, an external wall surface of the housing is cylindrical.

In some embodiments, the internal wall surface of the rotatable valve element is cylindrical.

In some embodiments, the slot is a first slot and further comprising a second slot in the sidewall that varies in depth along a circumferential length of the second slot and terminates in a second opening in the sidewall.

In some embodiments, a rate of change in the depth of the second slot differs from a rate of change of the depth of the first slot.

In some embodiments, the first slot and the second slot are circumferentially spaced from one another so that the first slot overlaps with the housing opening when the second slot does not overlap with the housing opening and the second slot overlaps with the housing opening when the first slot does not overlap with the housing opening.

In some embodiments, a first part of the first slot is circumferentially spaced from the second slot and a second part of the first slot overlaps circumferentially with the second slot such that when the first part of the first slot overlaps with the housing opening the second slot does not overlap with the housing opening and when the second part of the first slot overlaps with the housing opening the second slot overlaps with the housing opening.

In some embodiments, the second slot is substantially parallel to the first slot, and wherein the first slot gradually deepens for a first number of rotational degrees, the second slot gradually deepens for a second number of rotational degrees, the second amount beginning at an end of the first slot.

In some embodiments, the second slot is substantially parallel to the first slot, and wherein the first slot gradually deepens for a first number of rotational degrees, the second slot gradually deepens for a second number of rotational degrees, the second number of rotational degrees beginning at the opening of the sidewall of the first slot.

In some embodiments, a second slot is substantially parallel to the first slot, wherein the first slot gradually deepens for a first number of rotational degrees until a first point at which the depth of the first slot remains constant, the second slot gradually deepening for a second number of rotational degrees, the second number of rotational degrees beginning from the first point at which the depth of the first slot remains constant or from a later point during which the first slot remains at a constant depth.

In some embodiments, a width of the first slot and of the second slot are substantially identical.

In some embodiments, a width of the first slot and of the second slot are different.

In some embodiments, a circumferential length of the opening in the sidewall of the first slot differs from a circumferential length of the opening in the sidewall of the second slot.

In some embodiments, the first slot is in a different axial position from the second slot.

In some embodiments, the first slot and the second slot are not parallel to one another.

In some embodiments, a rate of change of the depth of the first slot differs from a rate of change of the depth of the second slot.

In some embodiments, the first slot does not change in width along the circumferential length of the first slot and the second slot changes in width along the circumferential length of the second slot.

In some embodiments, wherein the first slot is configured to provide a range of gas flow rates suitable for adults and the second slot is configured to provide a range of gas flow rates suitable for neonates.

In some embodiments, the valve further comprises a third slot in the sidewall that varies in depth along a circumferential length of the third slot and terminates in a third opening in the sidewall. In some embodiments, each of the first slot, the second slot and the third slot has a different width.

In some embodiments, the first slot and second slot are part of a series of two or more slots in the sidewall in which each slot of the series terminates in an opening in the sidewall and differs in regard to one or more of (i) slope (ii) a width or a change of width, (iii) circumferential length (iv) circumferential length of the opening of the slot.

In some embodiments, at least two of the first slot, the second slot and the third slot have a substantially same slope.

In some embodiments, at least two of the first slot, the second slot and the third slot have a substantially same circumferential length.

In some embodiments, at least two of the first slot, the second slot and the third slot have a substantially same rate of change of width.

In some embodiments, at least two of the first slot, the second slot and the third slot have a substantially identically shaped profile.

In some embodiments, each of the first slot, the second slot and the third slot has a different slope.

In some embodiments, each of the first slot, the second slot and the third slot has a different circumferential length.

In some embodiments, each of the first slot, the second slot and the third slot has a different rate of change of width.

In some embodiments, each of the first slot, the second slot and the third slot has a differently shaped profile.

In some embodiments, the housing opening is a first housing opening and further comprising a second housing opening extending from the interior passage through the housing, wherein a circumferential position of the first housing is different than a circumferential position of the second housing.

In some embodiments, the housing opening is a first housing opening and further comprising a second housing opening extending from the interior passage through the housing, wherein an axial position of the first housing is different than an axial position of the second housing.

Another embodiment is a gas control system for ventilating patients, comprising a source of gas; the variable orifice rotary valve of any of the versions of the variable orifice rotary valve; and at least one flow sensor and at least one pressure sensor. In some embodiments, the system further comprises a processing unit that includes a processor and programmable instructions or software stored on a tangible or non-transitory computer-readable medium, the programmable instructions or software, when executed by the processor, configured to control the at least one flow sensor and at least one pressure sensor.

Another embodiment is a method of operating a medical ventilator having a valve in which a valve element comprising slots rotates with a housing, the method comprising rotating the valve element such that a first slot is configured to provide a range of gas flow rates suitable for neonates without making use of the second slot and such that as the valve element is rotated the depth of the first slot, aligned with a housing opening, varies; and further rotating the valve element such that a wider second slot is configured to provide a range of gas flow rates suitable for adult without making use of the first slot, wherein the first slot and the second slot are circumferentially spaced from one another.

### BRIEF DESCRIPTION OF THE DRAWINGS

Various embodiments are herein described, by way of example only, with reference to the accompanying drawings, wherein;

FIG. 1a is an isometric view of a housing of a variable orifice rotary valve showing a housing opening on its side, in accordance with one embodiment;

FIG. 1b is a closed position of a variable orifice rotary valve, in accordance with one embodiment;

FIG. 1C shows a valve element whose slot is starting to be seen through the housing opening of the housing, in accordance with one embodiment;

FIG. 1D shows the valve element of FIG. 1C further rotated so that the slot allows a larger opening overlapping with the housing opening of the housing, in accordance with one embodiment;

FIG. 1E is a view as in FIG. 1D but showing the housing more clearly in the background, in accordance with one embodiment;

FIG. 2 is a partially opened position of a variable orifice rotary valve, in accordance with one embodiment;

FIG. 3a is a fully opened position of a variable orifice rotary valve, in accordance with one embodiment;

FIG. 3b is a partially opened position of a variable orifice rotary valve revealing a wider slot than in FIG. 3a, in accordance with one embodiment;

FIG. 4 is an isometric view of a rotatable valve element of a variable orifice rotary valve showing a slot therein, in accordance with one embodiment;

FIG. 5 is a sectional view of the rotatable valve element of a variable orifice rotary valve showing a profile of a thin slot therein, in accordance with one embodiment;

FIG. 6 is a sectional view of a rotatable valve element of a variable orifice rotary valve showing a profile of a thinner first slot within a first half of the circumference and a profile of a wider second slot within a second half of the circumference, in accordance with one embodiment;

FIG. 7a is a sectional view of the rotatable valve element of a variable orifice rotary valve showing a first portion of a wider slot on the top with a different slope than a second portion of that slot, and also showing a thinner slot on the bottom, in accordance with one embodiment;

FIG. 7b is a sectional view of the rotatable valve element of a variable orifice rotary valve from a different angle than in FIG. 7a and showing a first portion of a first slot therein with a different slope than a second portion of the first slot, in accordance with one embodiment;

FIG. 7C is an isometric view of a valve element with a different slope in a middle area of the slot, in accordance with one embodiment;

FIG. 8a is an isometric view of a rotatable valve element of a variable orifice rotary valve showing a thinner first slot and a wider second slot at different circumferential positions, in accordance with one embodiment;

FIG. 8b is an isometric view, taken 180 degrees rotational angle from that of FIG. 8a, of the rotatable valve element of a variable orifice rotary valve showing the thinner first slot and the wider second slot of FIG. 8a at different circumferential positions, in accordance with one embodiment;

FIG. 8c is an isometric view of a rotatable valve element of a valve showing a thinner first slot and a wider second slot at different axial positions, in accordance with one embodiment;

FIG. 9 is an isometric view of a rotatable valve element showing a thinner first slot, a wider second slot and a still wider third slot, in accordance with one embodiment;

FIG. 10a is an isometric view of two slots of the same width but starting at different rotational angles, in accordance with one embodiment;

FIG. 10b is an isometric view of the two slots of FIG. 10a as seen from a different angle and occupying a different location along the circumference of the valve element, in accordance with one embodiment;

FIG. 10c shows the slots of FIGS. 10a-10b through the housing opening of a valve at one exemplary location along the circumference of the valve element, in accordance with one embodiment;

FIG. 10d is an isometric view of two slots of the same width starting at different rotational angles together with the housing, in accordance with one embodiment;

FIG. 10e is an isometric view of two slots in a valve element within the housing where one of the slots has two areas of different slopes, in accordance with one embodiment;

FIG. 10f is an isometric view of a valve element showing two axially separated slots, one of which increases in width as its depth increases;

FIG. 10g is an isometric view of a valve element with a slot having a wavy profile, in accordance with one embodiment;

FIG. 10h is an isometric view of a valve element with a slot having a wavy profile inside a housing in accordance with one embodiment;

FIG. 11 is a schematic illustration of a gas flow system, in accordance with one embodiment; and

FIG. 12 is a flow chart showing a method, in accordance with one embodiment.

### DETAILED DESCRIPTION OF THE EMBODIMENTS

Certain embodiments generally provide a valve, which in some versions may be used to control the flow of respiratory gas (e.g., air, oxygen-enriched air, or oxygen) for example through a medical ventilator to deliver a desired pressure and/or volumetric flow rate to a patient. More generally, it should be appreciated that the valves disclosed herein, according to some embodiments, have numerous applications for controlling the flow of any gas(es). Only for simplicity purpose, the following disclosure will assume that the gas flowing though the valve is air. Accordingly, the following disclosure should not be considered limiting insofar as it focuses on applications for controlling airflow.

Aspects of the invention find a wide variety of applications in both medical and non-medical fields. Within the medical field, aspects of the invention are applicable to medical ventilators as well as to other medical applications in which flow of any gas is needed (air being one non-limiting example of such a gas). By way of one non-limiting set of implementations, the valve will be illustrated herein primarily in the context of medical ventilators. The figures illustrate one exemplary embodiment of a variable orifice gas flow control valve for example in the context of a medical ventilator application. It will be appreciated that this example is only one of a large number of suitable applications for the technology, as will be clear to a person of ordinary skill in the art. Accordingly, the valves, systems and methods disclosed herein are not limited to a medical ventilator application. They may be used in a variety of other applications, including, without limitation, mixing a plurality of gases, controlling flow in a vehicle heating/cooling system, etc.

In the medical ventilator field, and in particular for blower type medical ventilators, it is sometimes necessary to ramp up the amount of gas flow sometimes from one flow level to a different flow level. The flow rate sometimes needs to be adjusted fast and sometimes it needs to be adjusted slowly and with varying ranges and with very good resolution. This may be done to immediately increase the flow rate after the patient's breath is initiated so as to allow the lungs to be filled with less work of breathing by the patient. It is also necessary to fine tune the gas flow in response to the patient's condition and the settings of the ventilator. This may require frequent adjustments in the flow rate and target flow rates that are precise.

In the medical ventilator field for blower type medical ventilators, a blower generates a flow of air, oxygen or another gas (or a mixture of gasses) and the gas is pressurized to a low pressure, typically no more than 1 psi. In some ventilators, a valve in the pathway of the blower controls how much gas mixture or air or another gas flows into the patient. While for adults, a flow of 100 to 200 liters per minute may be needed in certain cases, for small neonates (as opposed to larger neonates), a flow of as little as 0.2 liters/minutes may be needed, which require more precise control. The variable orifice valve herein is configured to provide very gradual physical changes in the size of the slot carved into the valve element, and in particular in the depth of the slot. This provides a very high degree of control of the amount of flow and does so over a very wide range of flow rates. This is important in order to provide the precise gas flow rate to the patient in accordance with the patient's size, condition and state of breathing.

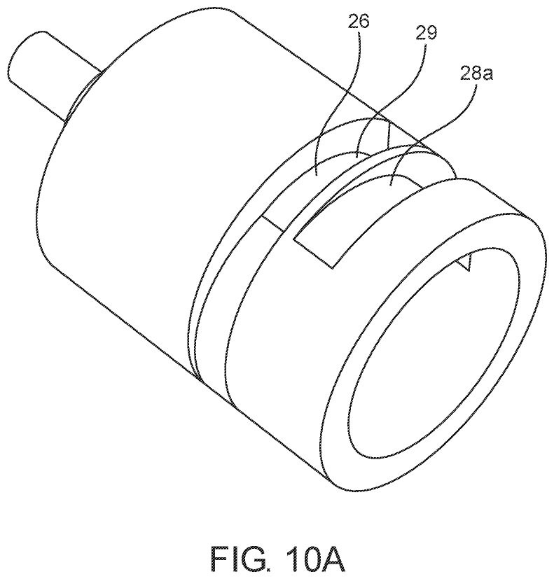

Using depth as a varying parameter, it is possible to exert a very high degree of control over the amount of the flow by making the changes in the depth of a slot of the valve element sidewall 22 (for example the depth of a first slot 26 or of a second slot 28a (FIG. 9) or of a third slot 28b (FIG. 9)) occur very gradually over many rotational degrees and by choosing a particular width of the slot or slots. The width of the slot(s) enables selection of the total required flow range, so that for example in some embodiments a wide slot such as slot 28a will be used when an adult is being ventilated and the thin slot such as slot 26 will be used for neonates. Furthermore, using electrical control over the rotation of the valve element (for example using a stepper motor's shaft connected to the valve element), it is possible to rotate the valve element by very small increments of a single rotational degree. If for example the increments are 1/32 of a rotational degree, there are over 10,000 different positions of rotation for 360 degrees.

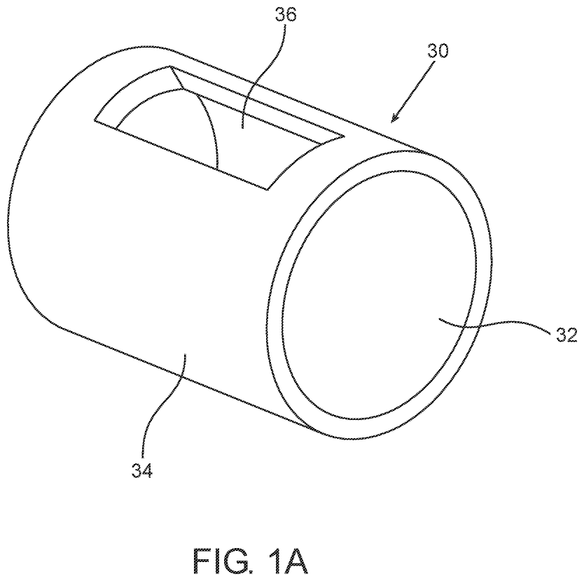

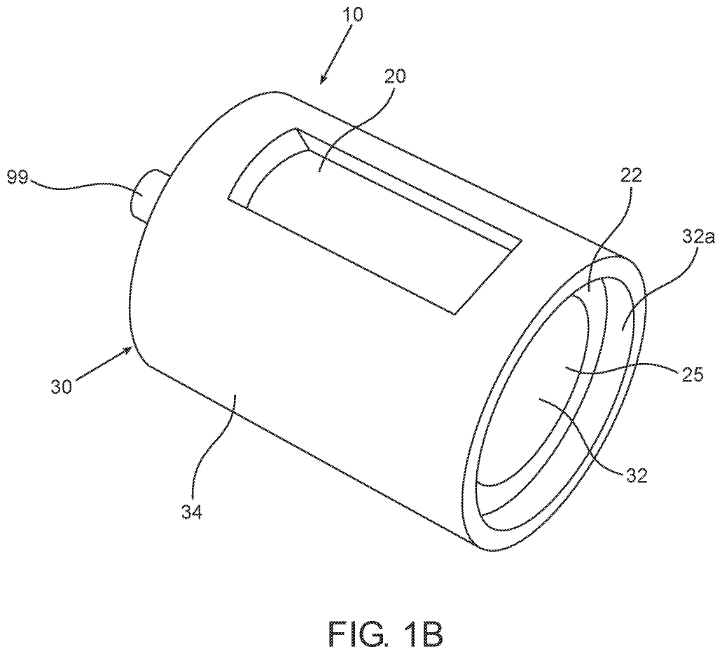

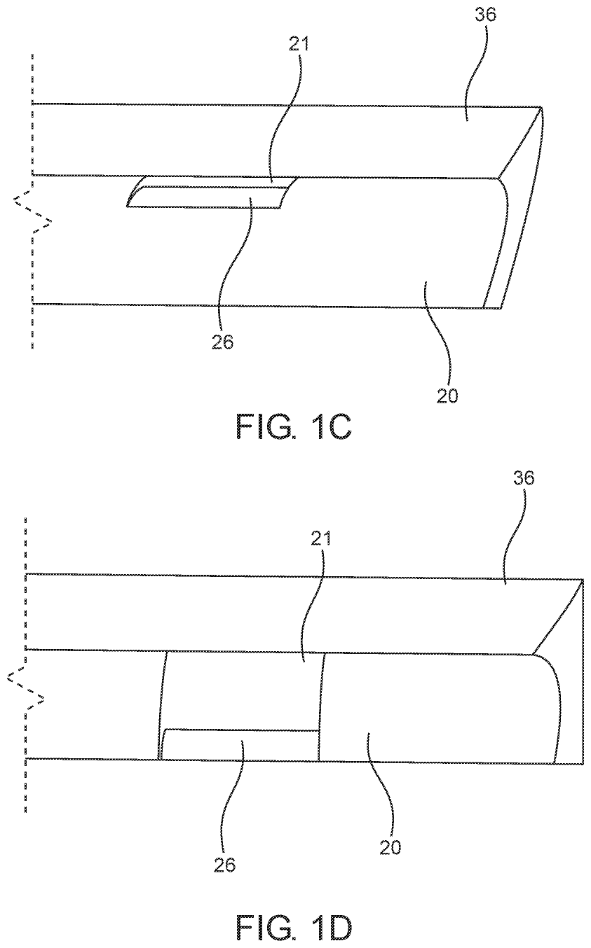

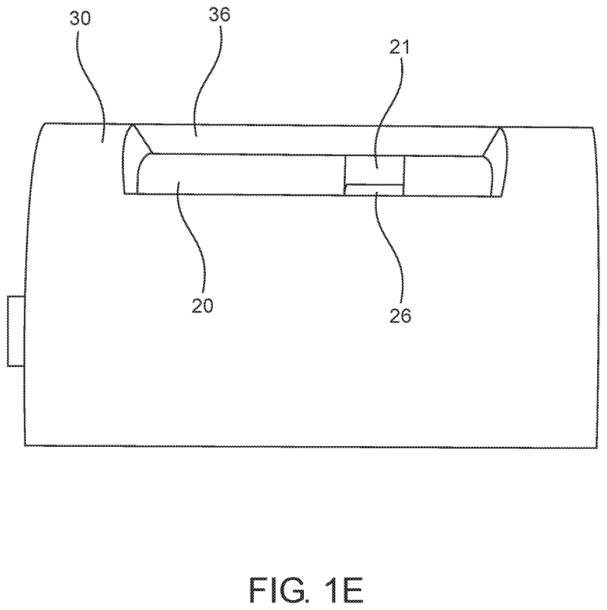

Typically, the slot changes in depth gradually. FIG. 1C and FIG. 1D show different stages in the rotation of the valve element 20 within housing 30, As the rotation of the valve element causes the slot (whether a first slot 26, a second slot 28a or a third slot 28b (FIG. 9) etc.) of valve element 20 to be aligned with the housing opening 36 of the housing 30 such that there is complete or partial overlap between them (and to the degree that the rotation of the valve element 20 causes such alignment of the slot with opening 36), the change in depth of the slot translates into a change in the gas (or other fluid) flow rate out of the valve 10. In FIG. 1D and FIG. 1E, where the valve element 20 has rotated further within housing 30, more of the slot 26 overlaps with the housing opening 36 of housing 30 and the slot space 21 (dark area) of the slot 26 is thicker. The term “overlap” therefore refers not only to the fact that rotationally the housing opening 36 overlaps partially or completely with a particular slot (26, 28a, 28b etc.) on the valve element 20, but also to the fact that the depth of such slot is such (as a result of at least some diminution of the thickness of the sidewall 22 of valve element 20) that it defines a gap between the sidewall 22 of the valve element 20 and the inner wall surface 32a of housing 30. As explained below, that space is in communication with an opening of the sidewall where the slot terminates such that the slot forms part of a fluid channel (for example gas channel) for the gas to flow from the valve opening through the sidewall 22 and through the housing opening 36. Although the fluid discussed explicitly herein is a gas or a mixture of gases, since that is the fluid commonly used in medical ventilators, it should be understood that the invention is applicable to fluids other than gases and that the fluid channel described herein may also refer to a channel of liquid fluid.

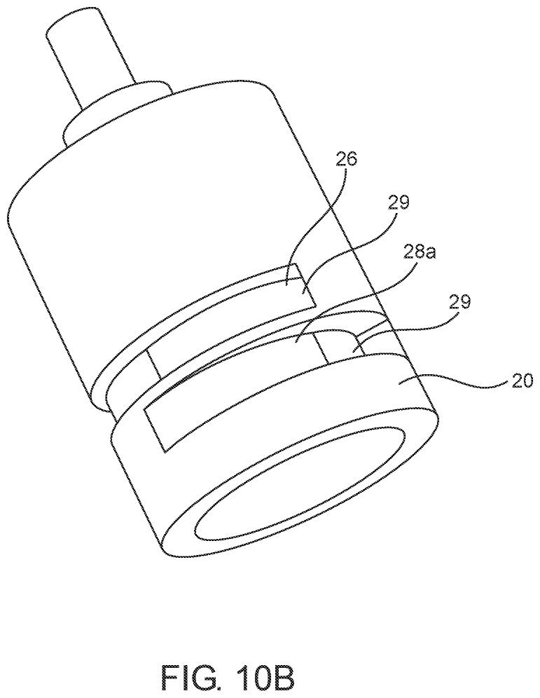

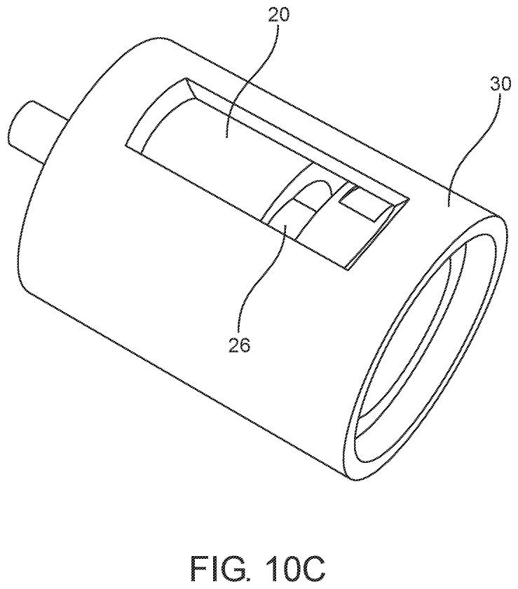

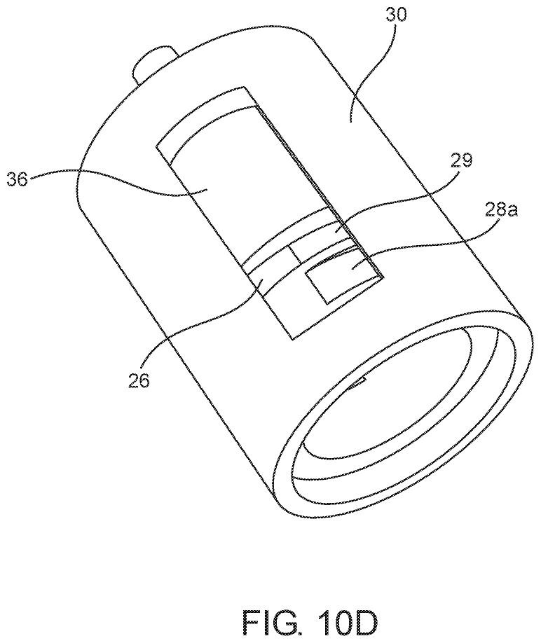

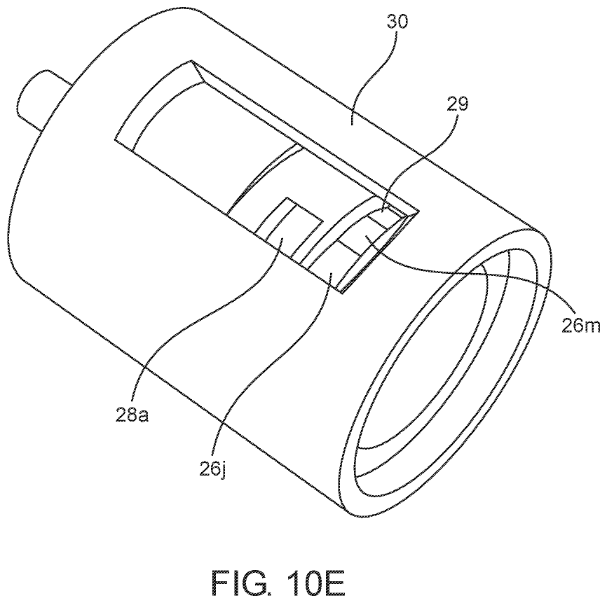

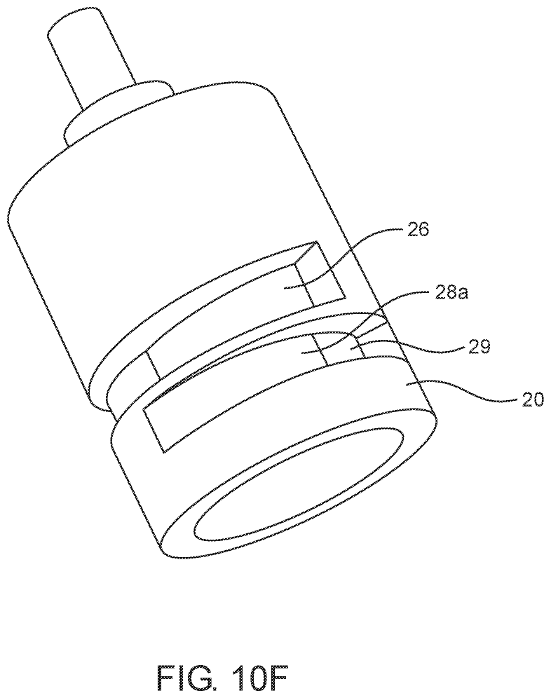

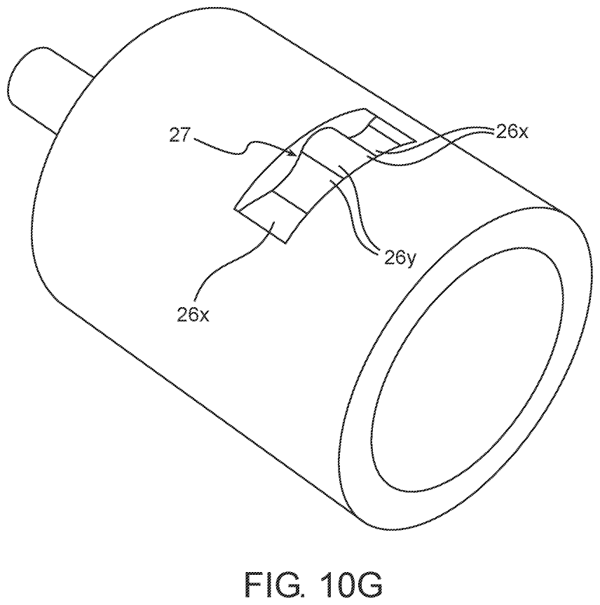

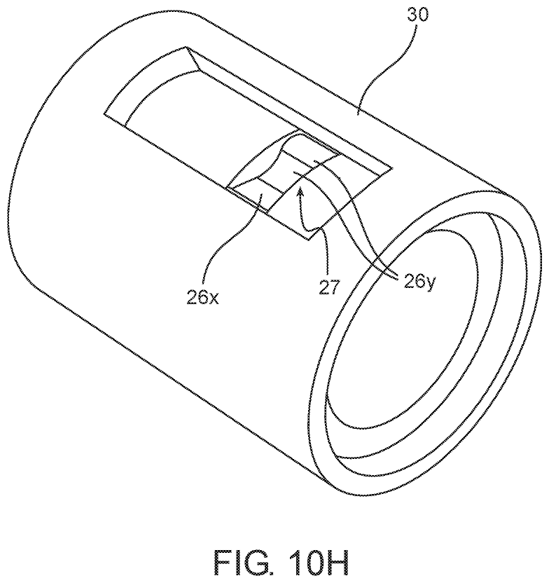

The change in depth of the slot may occur for example over any number of rotational degrees, for example over 20 rotational degrees, over 40, over 60, over 90, over 135, over 180, over 270 or over 300 rotational degrees or over any number of rotational degrees in between. The change in depth of the slot can be linear or nonlinear. In one scenario shown in FIG. 10a and FIG. 10b and in FIG. 10d and FIG. 10e, a first slot 26 changes in depth gradually over, for example, 140 to 160 rotational degrees so that the slot area that overlaps the housing opening is deeper and deeper providing more flow and then (as seen in FIG. 10d and FIG. 10e) over the next for example 140-160 rotational degrees the first slot is fully open (so that the flow through the first slot 26 does not increase anymore) and in addition a second slot 28a alongside or substantially parallel to the first slot 26 starts to overlap the housing opening 36 and gets deeper increasing the overall opening even more as it becomes deeper (along the overlap with housing opening 36) so as to enable a higher maximal flow. In this example for the first 140-160 rotational degrees the flow increases due to the first slot 26 and then for the next 140-160 rotational degrees the flow increases further due to the second slot 28a.

The second slot may begin alongside a particular part of the first slot. For example, this effect may be created from having second slot 28a begin at the opening of the sidewall of the first slot 26 such that the first slot 26 gradually deepens for a first number of rotational degrees, the second slot gradually deepening for a second number of rotational degrees that begins from an opening in the sidewall of the first slot 26. In some cases, as in FIGS. 10a-10b, the width of first slot 26 and the width of second slot 28a are substantially the same. In other cases, the respective widths of the first slot 26 and second slot 28a differ. In another implementation of this scenario, there are three slots (26, 28a, 28b) instead of two slots (26, 28a) and the third slot 28c begins at a terminal portion 29 of the second slot 28a or can be at any other location depending on the required flow profile required

Note that the reason that it is not practical to have a single slot stretch over a large number of rotational degrees, for example 280 to 320 to achieve that increased flow rate, is due to the limitations caused by the thickness of the sidewall 22 of valve element 20. Therefore, once the first slot 26 is so deep that the wall thickness of sidewall 22 is zero, the second slot 28a takes over the job of increasing the overall flow. It should be clear, moreover, as seen in FIG. 10a and FIG. 10b, that this effect is certainly possible even though the first slot 26 and the second slot 28a have the same width. That said, in some embodiments there can be a slot that extends, for example, a large number of rotational degrees, for example 320 degrees, but in that case the sidewall 22 would be thicker and the range of flow rates may be achieved by one slot.

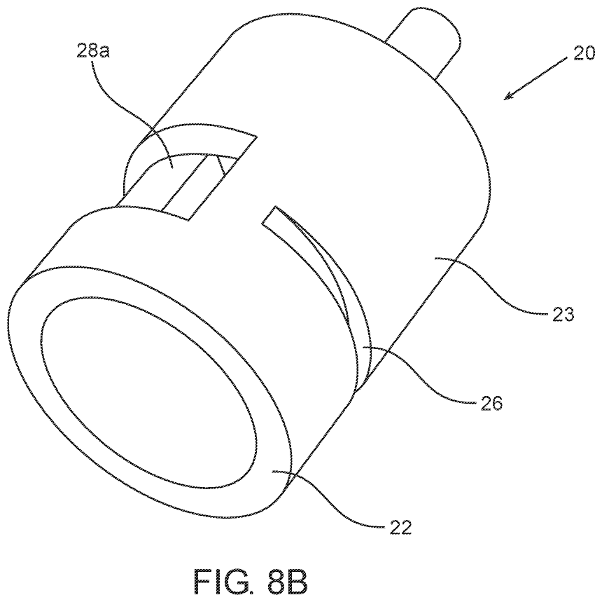

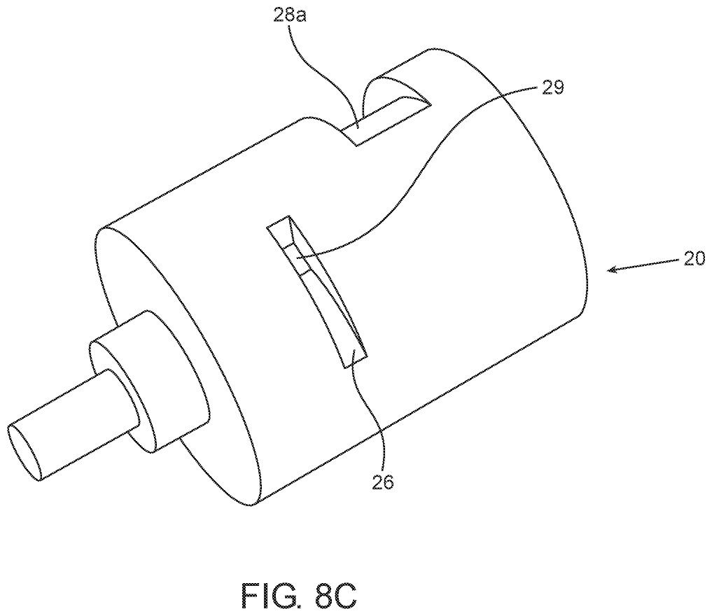

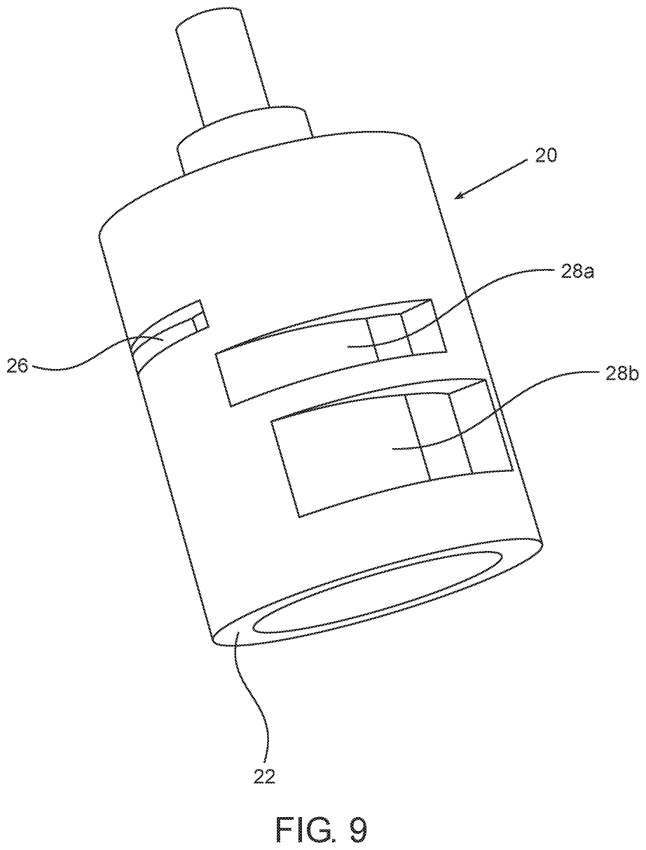

In another non-limiting example of a scenario shown in FIG. 8a and FIG. 8b, a thin first slot 26 changes depth gradually over for example about 140 rotational degrees (or any other number of rotational degrees) and then after a gap of about 40 or 60 rotational degrees (or a different number of rotational degrees) a second slot (which typically would be wider than first slot 26) appears on the other half of the circumference of the valve element (without any first slot 26) and changes in depth over a further about for example 140 rotational degrees (or any other number of rotational degrees). Using this last version, it is possible to have a thin slot that provides suitable flow rates (liters/minute) for small neonates and to have a thicker slot on the other half of the circumference that provides suitable flow rates for adult patients. In another implementation of this scenario, as shown in FIG. 9, there are three slots (26, 28a, 28b) instead of two slots (26, 28a) and each of the three slots (26, 28a, 28b) extends for example 80 rotational degrees with gaps of for example about 20-30 rotational degrees between them.

The above amounts of rotational degrees are merely non-limiting examples. Many other amounts of rotational degrees may be used. In addition, the steepness of the change in depth of the slot is another parameter that may be adjusted from one slot to another (or even within a single slot) to control the rate of flow of the gas, for example air.

Certain embodiments of the variable orifice rotary valve herein achieve some or all of the following objectives: i) provide fine control over the flow restriction for example due to the gradual changes in depth, the option to use multiple slots stretching over a larger rotational range and the availability of the differently sloped slots and in some case also slots of different widths; ii) provide relatively linear control of the flow when needed; iii) achieve fast response time from fully closed to fully open without excessive power consumption; iv) are easily controlled electronically since for example the changes in gas flow rate are achieved through simple rotation of one component; v) control flow over a very wide range of flow rates for example because there can be a change of depth and width or because multiple slots are used and they that enable control for different flow ranges vi) function without lubrication for example since a graphite (or other suitable material) valve element can rotate within a glass housing (or a housing of other suitable material) without lubrication; vii) in ventilation applications, for example involving blowers, enable control of flow of air or oxygen or a gas mixture to the patient, and viii) control flows at very low pressures, such as in ventilation applications, due to the ability to use a large size orifice opening by using a wide slot or slots yet at the same time retain the option to have a small overlap when necessary for low pressure.

The principles and operation of a Variable Orifice Rotary Valve for Controlling Gas Flow may be better understood with reference to the drawings and the accompanying description.

Turning now to the non-limiting example shown in FIGS. 1a-10c, these figures show a variable orifice rotary valve 10 for controlling gas flow. As shown in FIG. 1a, valve 10 may comprise a housing 30 including a substantially cylindrical interior passage 32 defined by a generally cylindrical inner surface 32a (FIG. 1b), and a housing opening 36 extending from the interior passage 32 through the housing 30. In one non-limiting example, housing 30 may have an external wall surface 34 that may be cylindrical. Although typically the housing 30 is cylindrical it may also be rectangular or have other shapes so long as the inner surface 32 of housing 30 is cylindrical (or substantially cylindrical). In addition, although housing opening 36 appears in the drawings as rectangular, it can assume other shapes as well. In certain embodiments, housing opening 36 is wide enough to span the width of the slot of the rotatable valve element that it is configured to overlap with. Housing 30 may be made of glass or of other materials.

As shown in FIG. 1b through FIG. 10c, valve 10 may further comprise a rotatable valve element 20 including a sidewall 22 having a cylindrical external sidewall surface 23 and an internal wall surface 24. In one non-limiting version, internal wall surface 24 of valve element 20 is also cylindrical or substantially cylindrical, although it can also be other shapes.

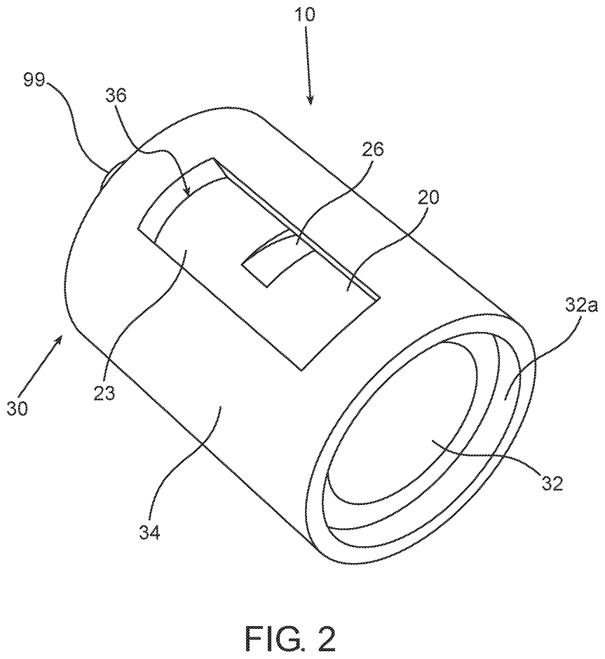

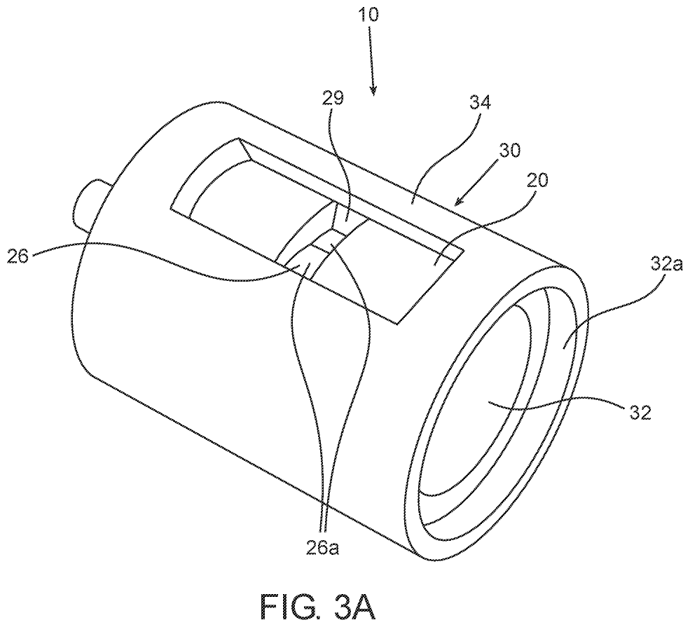

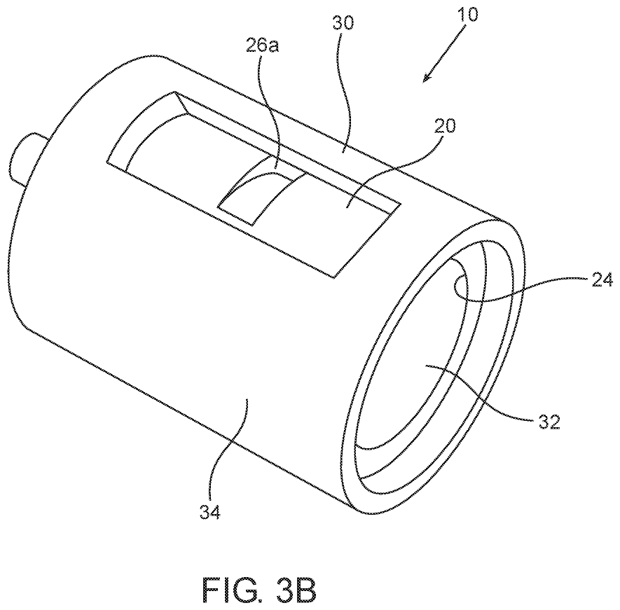

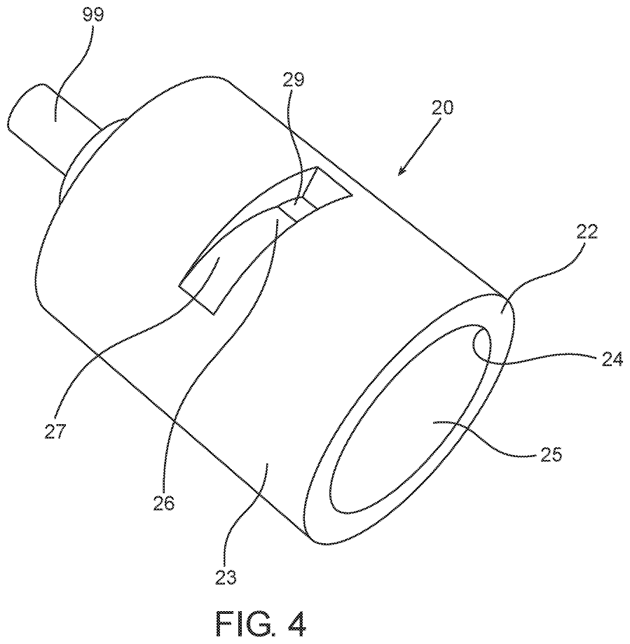

Valve element 20 has a main opening 25 (FIG. 4). As seen from FIG. 2, FIG. 3a and FIG. 3B, valve element 20 may be a cylindrical rotary valve element that is rotatably received within housing 30, and more particularly within the interior passage 32 of housing 30 such that a rotation of the valve element 20 within the interior passage 32 determines a circumferential length and a depth of a portion 26a of the slot (for example slot 26) that overlaps with the housing opening 36. As seen in FIGS. 2-4, the valve element 20, in certain embodiments, is typically hollow starting at main opening 25 and includes a side opening 26 (called a slot 26 or a first slot 26) forming part of a fluid channel, for example a gas path, from main opening 25 to first slot 26. The gas path may be from main opening 25 of the valve element 20 to and through the housing opening 36 such that main opening 25 acts as an inlet and the side opening (for example first slot 26) as the outlet or vice versa. The gas flow rate (or the flow rate of another fluid) through the fluid channel (for example a gas channel) is determined at least in part by the circumferential length and the depth of the portion 26a of the slot 26 (or any slot) that overlaps with the housing opening 36 (as well as by the width of the slot). Accordingly, the rate of gas flow through valve 10 varies with a rotational angle of the valve element 20,

Any of the slots, for example first slot 26 or slot 28a, in sidewall 20 has a three-dimensional structure as seen from the different positions of valve element 20 shown in FIGS. 4-6. As seen from FIGS. 4-6, a slot 26 in sidewall 22 (which may be referred to as a first slot 26 in situations where sidewall 20 has multiple slots) varies in depth along a circumferential length of the slot 26 so as to vary a thickness of the sidewall 22 along that circumferential length of first slot 26. The “circumferential length” of any slot along a surface 23 of the valve element 20 is not identical to the length of the slot floor surface 27 of the slot 26 although these two parameters are generally proportional. At some point along the circumferential length of the slot (and this applies to any slot 26, 28a, 28b etc.) the thickness of the sidewall 22 diminishes to where the sidewall 22 terminates in an opening 29 in sidewall 20. At the opening 29 of sidewall 22 of any slot, the depth of the slot 26 (relative to the external sidewall surface 23) is at its maximum.

The depth of the any slot (26, 28a, 28b, etc.) varies substantially along a direction of rotation of the valve element, which is the circumferential length of the slot. Typically, the change of depth of slot 26 is gradual such that the thickness of the sidewall 22 of valve element 20 is diminished gradually.

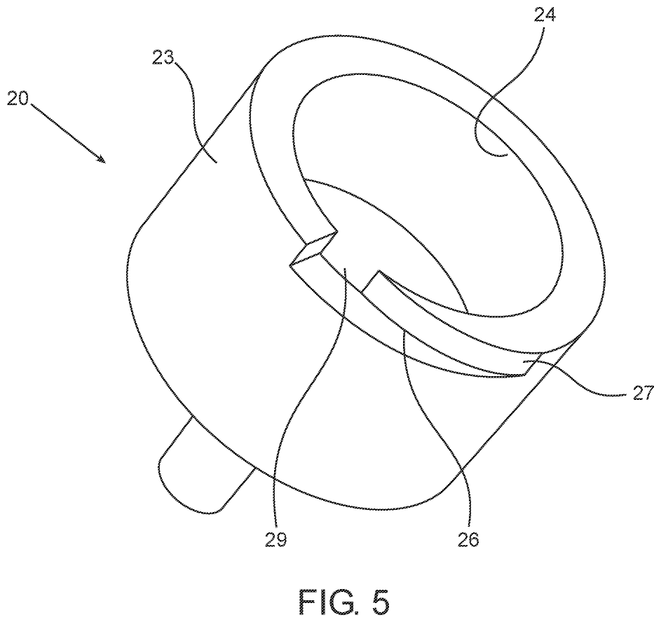

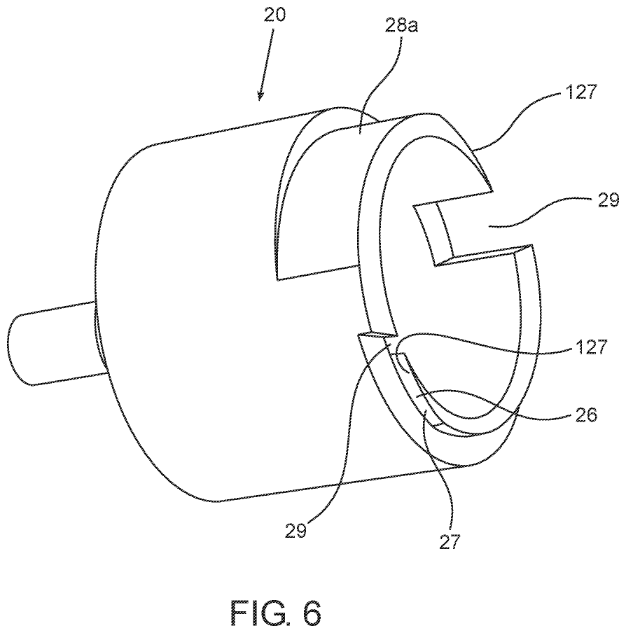

In some embodiments, as seen from FIGS. 5-6, the depth of the slot (whether first slot 26 or second slot 28a or any other slot) may vary nonlinearly throughout the circumferential length of the slot other than at the opening 29 of the slot. Accordingly, in this embodiment, where the depth varies nonlinearly, the rate of change of the depth of the slot 26 (or of any other slot 28a) may vary linearly (or at another rate) through at least a portion of the slot.

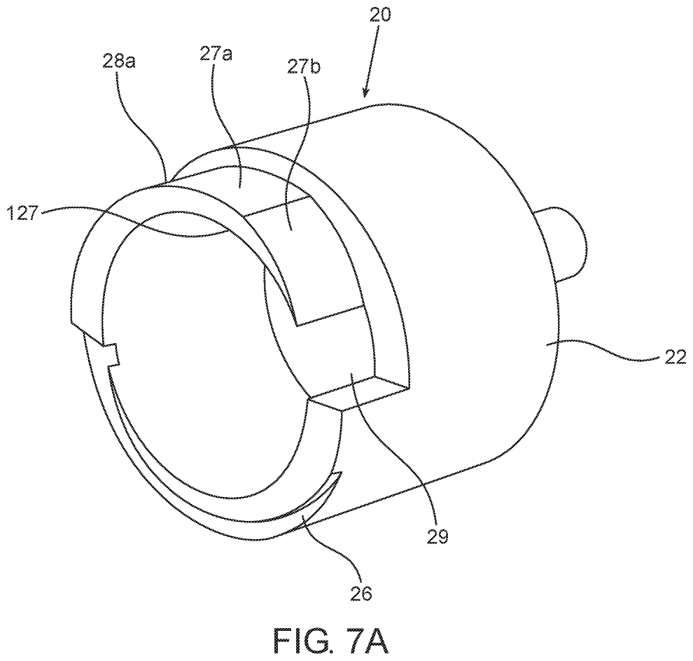

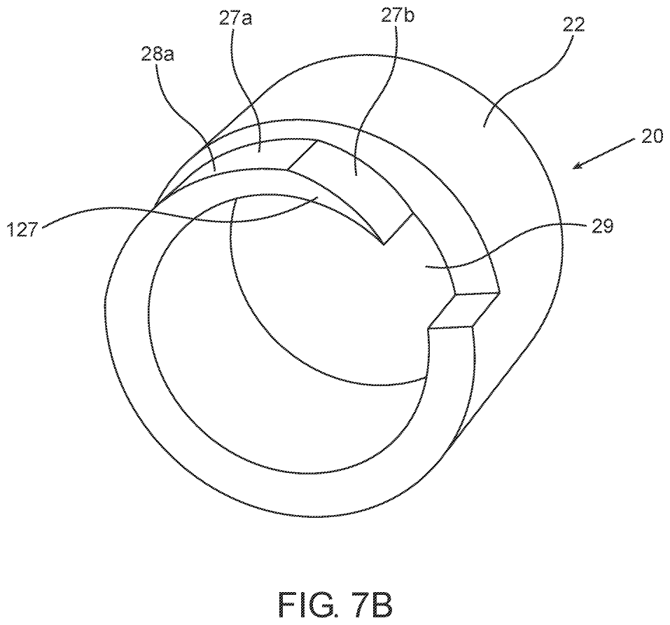

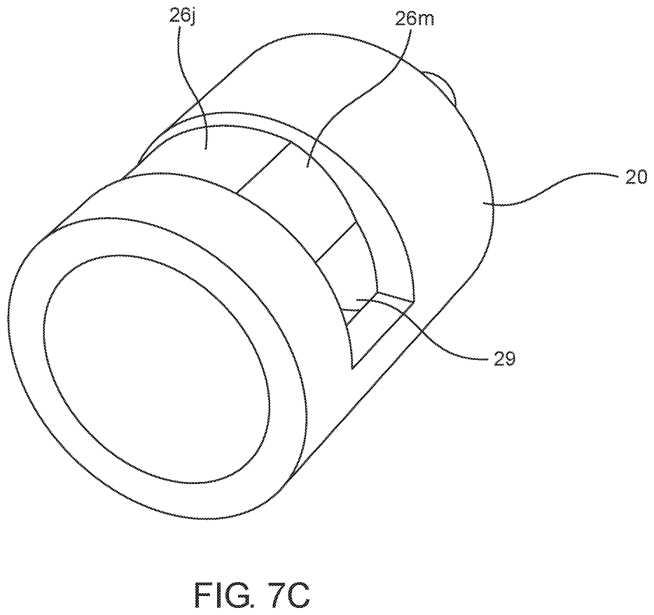

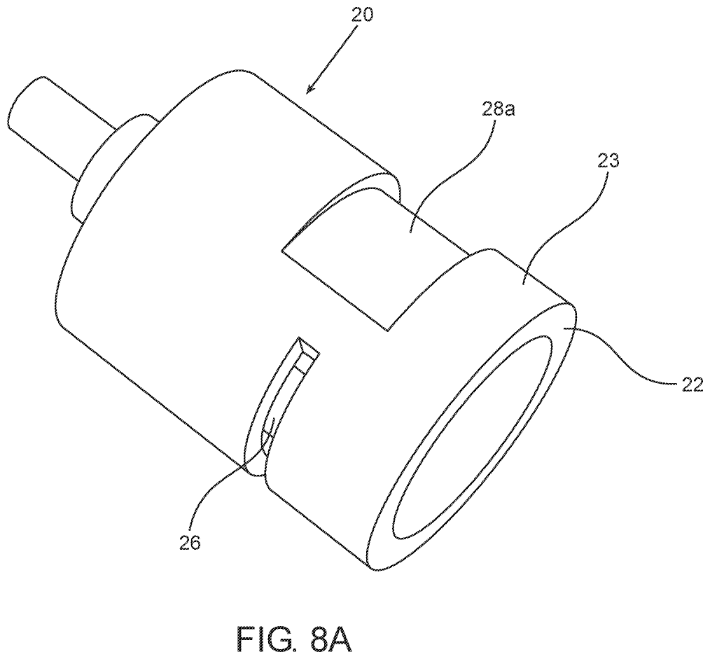

The rate of change of the depth of any particular slot (for example of the first slot 26 shown in FIG. 4, FIG. 5, FIG. 7a or of a second slot 28a shown in FIG. 6, FIG. 7a, FIG. 10a, FIG. 10b or of a third slot 28b shown in FIG. 9, etc.) may be controlled by using a particular steepness in the slope of the top surface 27 of the slot (first slot 26 or second slot 28a or third slot 28b, etc.). For example, as seen in FIG. 7a and FIG. 7b, the depth of the second slot 28a may vary (linearly or nonlinearly) through a first portion of the second slot 28a at a first steepness 27a and then may vary (linearly or nonlinearly) through a second portion of the second slot 28a at a second steepness 27b (more steep or less steep). In FIG. 7C, for example, there is a different slope in the second area 26m of the slot 26 indicating a different rate of change of depth compared to along the first area 26j of the slot. In this scenario the rate of change in any particular area of the slot may be linear or nonlinear.

In general, a “portion of a slot” refers to a portion of a circumferential length of the slot. Furthermore, references herein to a first portion and a second portion of a slot are, unless otherwise indicated, arbitrary with respect to direction and do not necessarily mean that the first comes before the second portion from a particular circumferential direction.

In general, the rate of change in the depth of a slot may change in a linear or nonlinear manner at a certain rate for part of the circumferential length of the slot and then the rate of change in the depth may decline or increase in other parts of the circumferential length of the slot. In some cases, the depth may not change at all for some portion of the circumferential length of the slot. Virtually any other combination ((i) linear followed by nonlinear variation in depth or (ii) nonlinear followed by linear variation in depth or (ii) linear or nonlinear variation followed by no variation in depth etc.) is possible—the above are only examples. Reference here to no variation in depth is to a portion of the slot bounded by the slot floor surface 27 (i.e., not the opening 29). In certain scenarios, portions of a slot in which there is no variation in depth may occur at any point—for example the slot may have a deepening portion (i.e., a downward slope) followed by a flat portion (no variation in depth) following by a further deepening portion.

In some embodiments, as in FIG. 10g (valve element 20 without housing 30) and FIG. 10h (valve element 20 together with housing 30) the depth of the slot increases (linearly or nonlinearly) (when viewing the figure from left to right) through a first portion 26x of the slot and then decreases (linearly or nonlinearly) through a second portion 26y of the slot typically such that the first portion 26y (deepening portion) is closer to the opening 29 of the slot than the second portion 26x (the portion where the depth is becoming shallower). The first portion or second portion of a slot may refer to a first portion or second portion of a circumferential length of the slot and there may be more than first portion (deepening portion) 26x and there may be more than second portion 26y (portion where the depth is becoming shallower). For example, as seen in FIG. 10g (valve element 20) and FIG. 10h (valve element 20 inside housing 30), the depth of any particular slot may reverse itself along the circumferential length of the slot at least once so as to present a wavy profile of the slot floor surface 27 of the slot. This embodiment is most likely to be useful in non-ventilator applications of valve 10 but is also useful in at least one ventilator application, for example, if a neonate needs repeated short pulses of air, oxygen or another gas. In that case, high frequency oscillations in the gas flow are achieved by rotating the valve element 20 within the housing 30 across multiple waves of the top surface 27 of the slot 26.

As seen from FIGS. 1a-11b, in some implementations, a change of the depth of any of the slots (26, 28a, 28b etc.) can occur through any circumferential length of the slot. In some non-limiting examples, the change in depth can occur through 20 rotational degrees, 30, 40, 45, 50, 60, 70, 80, 90, 120, 135, 150, 180, 270, 280, 290, 300, 310, 320, 330 or any number in between these examples or any range between any two of these numbers or other number in between these numbers. Accordingly, the size (length, width and depth) of any slot (i.e., 26, 28a, 28b etc.)—and therefore, the flow rate of the gas flowing through valve 10, can be adjusted by rotation of valve element 20 through a wide range of rotational angles (for example about 300° or more of rotation), enabling very fine adjustment for example from relatively small changes in angle of rotation of the valve element 20.

FIG. 1b shows a closed position of variable orifice rotary valve 10, in accordance with one embodiment, wherein there is no overlap (i.e., no alignment) of a slot with housing opening 36 and therefore no slot of valve element 20 is visible in the drawing. FIG. 2 (a partially opened position of the variable orifice rotary valve 10 shown in FIG. 1b), in contrast, shows the valve 10 after there has been some rotation of valve element 20 (for example by an electrical controller that controls a shaft 99 connected to valve element 20) to a different rotational angle than that shown in FIG. 1b. Consequently, in FIG. 2 there is some overlap that can be seen between slot 26 and housing opening 36. After further rotation of valve element 20, however, as seen in FIG. 3a, valve 10 is now in a fully opened position. FIG. 3b shows another partially open position of valve 10 with a longer overlapping portion 26a of a somewhat wider slot than that shown in FIG. 2.

Slot 26 extends around a portion of the circumference of valve element 20, and, in particular, the circumference of cylindrical external sidewall surface 23 of valve element 20. Due to the change in depth along the length of the slot 26, as valve element 20 is rotated, the maximum depth of the portion 26a of slot 26 that overlaps with housing opening 36 continually increases.

When slot 26 is rotated so that it does not overlap with housing opening 36 in housing 30, the valve 10 is in the closed position (FIG. 1b) since slot 26 is blocked from view. However, by rotating valve element 20 in housing 30, the length of slot 26 that overlaps with housing opening 36 becomes longer and the slot 26 becomes deeper in the sense that it has a greater maximum depth facing the housing opening 36. Therefore, the slot 26 (combined with housing opening 36) provides valve 10 with a variable orifice.

As noted, slot 26 in sidewall 22 may be a first slot and sidewall 20 have more than one slot such as second slot 28a in sidewall 22 or third slot 28b in sidewall 22 etc. Second slot 28a may vary in depth along a circumferential length of the second slot 28a and terminate in an opening 29 and likewise third slot may vary in depth along a circumferential length of the third slot 28a and terminate in an opening 29.

In some embodiments, a rate of change in the depth of the second slot 28a differs from a rate of change of the depth of the first slot 26. In one implementation shown in FIG. 7a, the sidewall 22 has a second slot 28a. Each slot (26, 28a, 28b, etc.) may have its own shape and its own depth, length, area, size. For example, in FIG. 7a, the second slot 28a has a depth along its length that has a different rate of change than the rate of change of the depth of the first slot 26.

Within the same slot, moreover, the rate of change of the depth of the slot may vary. As seen in FIG. 7a and FIG. 7b, for example, the change in the depth of the slot (for example second slot 28a of FIG. 7a) during a first portion of the length of the second slot 28a, for example during a first 100° of rotation, can be relatively low (for example, going from 0 mm-1 mm) and then the change in the depth of the slot during a second portion of the circumferential length of the slot for example during the next 100° can be higher (for example, from about 1 mm-3 mm which is twice as fast as for the first 100°). These amounts of rotations (i.e., 100°) in a portion of the slot 28a are merely non-limiting examples that are larger than the actual ranges of rotation in the first or second portion of the length of slot 28a depicted in FIGS. 7a-7b. FIG. 7c also shows a rate of change of the depth varying within the same slot.

According to some embodiments, it is possible to rotate the valve element 20 using a very gently sloped slot 26 (or second slot 28a or third slot 28b etc.) whose depth varies very gradually. For example, it is possible to have increments in the gas flow rate of 0.1 liters per minute. It is also possible, within the same valve 10 to have a steeper slope, which is one way of generating increments in gas flow rate that are much higher (another way for example is by using multiple slots).

In addition, all of the versions or features described with respect to the slot in sidewall 22 apply as well to a second slot 28a, to a third slot 28b or to any other slot.

In some embodiments, the second slot (for example second slot 28a) is substantially parallel to the first slot 26, and the first slot gradually deepens for a first number of rotational degrees, and the second slot gradually deepens for a second number of rotational degrees, the second amount beginning at an end of the first slot. The “end” refers to the last point along the circumferential length of the slot which in this case would be the endpoint of the opening 29 of the slot 26.

In some embodiments, for example in FIG. 10a, the second slot is substantially parallel to the first slot, and wherein the first slot gradually deepens for a first number of rotational degrees, the second slot gradually deepens for a second number of rotational degrees, the second number of rotational degrees beginning at the opening 29 of the sidewall of the first slot 26. In FIG. 10a, the second number of rotational degrees begins at the beginning of the opening 29.

In another scenario, a second slot is substantially parallel to the first slot, wherein the first slot gradually deepens for a first number of rotational degrees until a first point at which the depth of the first slot remains constant, the second slot gradually deepening for a second number of rotational degrees, the second number of rotational degrees beginning from the first point at which the depth of the first slot remains constant or from a later point during which the first slot remains at a constant depth.

In some embodiments shown in FIG. 10a and FIG. 10b a width of the first slot and of the second slot are substantially identical. In another version shown in FIG. 6, FIG. 7a, FIG. 8a and FIG. 8b, a width of the first slot and of the second slot are different.

A circumferential length of the opening in the sidewall of one slot may differ from a circumferential length of the opening in the sidewall of a second slot.

As shown in FIG. 8c, FIG. 9, FIG. 10a and FIG. 10b, a particular slot may be in a different axial position that another slot in the sidewall 22.

Although the drawings show multiple slots as parallel or substantially parallel to one another, one slot and another slot in sidewall 22 may not be parallel or substantially to one another.

In some embodiments, as shown for example FIG. 6, FIG. 7a and FIG. 8c, a rate of change of the depth of the first slot 26 differs from a rate of change of the depth of the second slot 28a.

In some embodiments, as seen in FIG. 10f, the first slot does not change in width along the circumferential length of the first slot and the second slot changes in width along the circumferential length of the second slot.

In some embodiments, the first slot is configured to provide a range of gas flow rates suitable for adults and the second slot is configured to provide a range of gas flow rates suitable for neonates. For example, a wide slot such as slot 28a shown in FIG. 8a, FIG. 8b, FIG. 8c, will be used when an adult is being ventilated and the thin slot such as slot 26 will be used for neonates.

FIG. 6 is a sectional view showing a profile of a thinner first slot 26 located within a first half or other portion of the circumference (and having an upper surface 27) and a profile of a wider second slot 28a within a second half or other portion of the circumference. Note that “located within” a half of a circumference of valve element 20 means that it is situated somewhere within that half, not that it actually occupies that entire half of the circumference. In some embodiments, as seen especially in FIG. 6, a portion of the slot that varies on depth (which is equivalent to the portion of the sidewall 22 that varies in thickness) is curvilinear and has a curvilinear slot floor surface 27 such that a profile of the first slot 26 or second slot 28a shows sidewall 22 forming a tongue-like shape 127 or projection. However, alternative embodiments may have other profiles (for example a wavy profile). The change in depth in slot 26 is configured to provide fine control over the gas flow rate.

As best appreciated from the fully opened position of FIG. 3a and FIG. 3b, the portion 26a of the first slot 26 that overlaps with the housing opening 36 comprises part of a fluid channel (for example a gas channel) from the main opening 25 of the valve element 20 to and through the housing opening 36 (including the gas path within valve element 20). The larger the overlap between a slot (26, 28a, 28b etc.) and the housing opening 36, and the deeper the slot is within that overlap, the lower the resistance to the flow of gas (for example air) between the two openings (the slot and housing opening 36) since the overall opening (the slot 26 combined with housing opening 36) is enlarged. The overall opening can be said to form an outlet for the gas flow or an inlet.

A gas (or other fluid) flow rate through the fluid channel is determined at least in part by the length of slot (slot 26 or 28a or slot 28b, etc.) that overlaps with housing opening 36 and by the depth of the portion 26a of the first slot 26 (or the second slot 28a or the third slot 28b, etc.) that overlaps with the housing opening 36. The gas flow rate therefore varies depending upon the rotational angle of the valve element 20.

According to some embodiments of the valve 10, as shown in FIG. 5, FIG. 7a, FIG. 9, FIG. 10a and FIG. 10b, more than one slot (e.g., two or three or four or five or more) may be created in a different position either in the axial direction of valve element 20 (FIG. 8C, FIG. 9, FIG. 10a, FIG. 10b) or along the circumference (FIG. 8A, FIG. 8b). The width and the profile will define the control profile for the flow. According to some embodiments, the slots may have the same or different length, width, depth and/or depth gradient.

Multiple slots with different profiles may be positioned along the circumference of valve element 20 so that only one of the slots overlaps the housing opening 36 at any given time. When used in connection with a medical ventilator, this enables having one wider slot and one thinner slot, with the same or different depth profiles, supporting different flow rates—for example, one slot for an adult (high flow) and a different slot for a neonate (low flow). In some embodiments, the first slot and the second slot are circumferentially spaced from one another so that the first slot overlaps with the housing opening when the second slot does not overlap with the housing opening and the second slot overlaps with the housing opening when the first slot does not overlap with the housing opening. For example, in one version shown in FIG. 8a and FIG. 8b, the first slot 26 and the second slot 28a are circumferentially spaced from one another so that the first slot 26 overlaps with the housing opening 36 at a rotational angle at which the second slot 28a does not overlap with the housing opening 36 and the second slot 28a overlaps with the housing opening 36 at a rotational angle at which the first slot 26 does not overlap with the housing opening 36.

In another embodiment illustrated in FIG. 9 with respect to the second slot 28a and the third slot 28b, the first part of a slot (for example second slot 28a of FIG. 9) is circumferentially spaced from another slot (for example third slot 28b) but a second part of the slot (for example second slot 28a) does overlap circumferentially with the other slot (for example third slot 28b). In that case, when the first part of the slot (for example slot 28a) overlaps with the housing opening 36 the other slot (in this case slot 28b) does not overlap with the housing opening and conversely when the second part of the slot (in this case slot 28a) does overlap with the housing opening 36 the other slot (in this case slot 28b) does overlap with the housing opening 36.

According to some embodiments, the valves disclosed herein create numerous advantages. For example, the valves enable extremely flexible control. According to some embodiments, the sizes of the slot (i.e., cutout) in the sidewall 22 and the corresponding opening 36 in the housing determine the minimum flow restriction (maximum flow rate), enabling very low pressure drop at high flow rates. According to some embodiments, the shape of the slot (e.g., profile of length, width and depth) determines the relationship between the change in flow rate and the change in rotational angle of the rotatable valve element 20. For example, the effective area of the slot, and thus the flow rate through the opening at a given pressure, may vary (for example linearly or nonlinearly) with the rotational angle of the cylinder

According to some embodiments, the slot width may be modified to provide for additional control. According to some embodiments, as seen regarding slot 26 in FIG. 10f, the one or more slots (26, 28a, 28b, etc.) may have a varying width. For example, a particular slot or slots may be 3 mm wide for 100 degrees of rotation and then gradually widen for example at the same time that the slot (26, 28a, 28b etc.) becomes deeper. Accordingly, in one particular implementation of first slot 26, or of one of the slots (26, 28a, 28b, etc.), in at least a portion of the slot (26 or 28a or 28b etc.) a width of the particular slot (26, 28a, 28b, etc.) varies as the depth of that particular slot (26 or 28a or 28b etc.) varies, as seen in FIG. 10f.

In some embodiments, as shown in FIG. 9, there is a third slot 28b in the sidewall 22 that varies in depth along a circumferential length of the third slot 28b and terminates in an opening 29 (referred to here as a third opening 29) in the sidewall 22. In some embodiments, as shown in FIG. 9, each of the first slot 26, the second slot 28a and the third slot 28b has a different width.

In general, the first slot and second slot may be part of a series of two or more slots in the sidewall 22 in which each slot of the series terminates in an opening in the sidewall 22 and differs in regard to one or more of (i) slope (ii) a width or a change of width, (iii) circumferential length (iv) circumferential length of the opening of the slot.

In some embodiments, at least two of a first slot, a second slot and a third slot has a substantially same slope. In some embodiments, at least two of the first slot, the second slot and the third slot have a substantially same circumferential length. In some embodiments, at least two of a first slot, a second slot and a third slot have a substantially same rate of change of width. In some embodiments, at least two of the first slot, the second slot and the third slot have a substantially identically shaped profile.

In some embodiments, each of a first slot, a second slot and a third slot has a different slope. In some embodiments, each of the first slot, the second slot and the third slot has a different circumferential length. In some embodiments, each of the first slot, the second slot and the third slot has a different rate of change of width. In some embodiments, each of the first slot, the second slot and the third slot has a differently shaped profile.

Each of the above scenarios also applies to a valve element 20 having more than three slots.

In some embodiments of housing 30, and this relates to any of the scenarios of the number or shape or position etc. of the slots discussed herein, there is more than a single housing opening 36. For example, in one embodiment, housing opening 36 is a first housing opening and housing 30 further includes a second housing opening (not shown) extending from the interior passage 32 through the housing, wherein at least one of the following is true (i) a circumferential position of the first housing is different than a circumferential position of the second housing and (ii) an axial position of the first housing is different than an axial position of the second housing. In one version, the second housing differs from the first housing opening 36 as to a positional orientation of the housing opening (i.e., the extent to which the sides of the housing opening run along the circumferential length of the housing 30).

Furthermore, valve 10 may also comprise any combination of the features described herein. For example, valve 10 may include multiple slots (26, 28a, 28b etc.) and multiple housing openings 36 or valve 10 may include one or more changes in the slope and/or width and/or depth and/or circumferential length (and/or other parameter such as but not limited to axial or circumferential spacing between slots or such as orientation (whether parallel or non-parallel)) of one or more slots of valve element 20 of valve 10 with one or more housing openings 36.

When used in connection with a medical ventilator, the valve 10 illustrated herein is not likely to be used at high pressures. Thus, selection of proper materials enables low inertia, fast response and very long life without lubrication. For example, in one embodiment, the housing 30 may be glass, or may include a glass lining in the housing 30, and the rotatable valve element 20 may be graphite. Both glass and graphite are inexpensive materials, and they have matching temperature coefficients. In that case, temperature will have a small effect on tolerances between valve element 20 and housing 30, keeping the air leak negligible while maintaining no friction and wear. However, in other embodiments, different materials can be used.

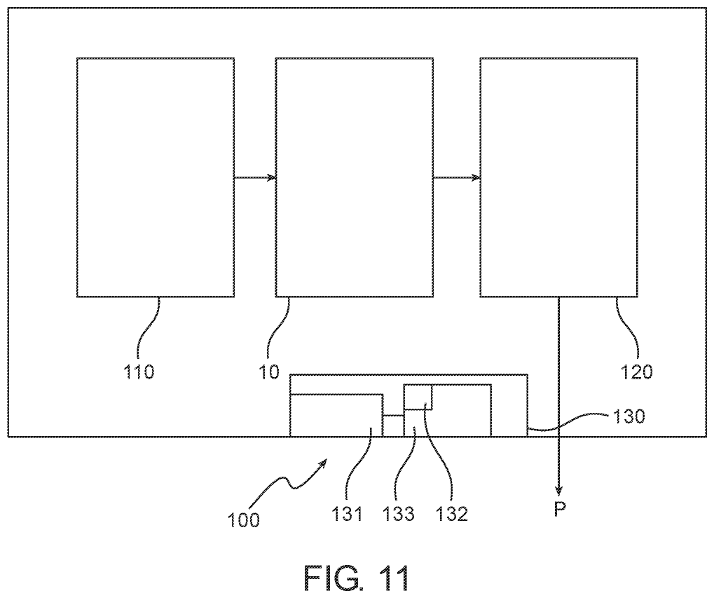

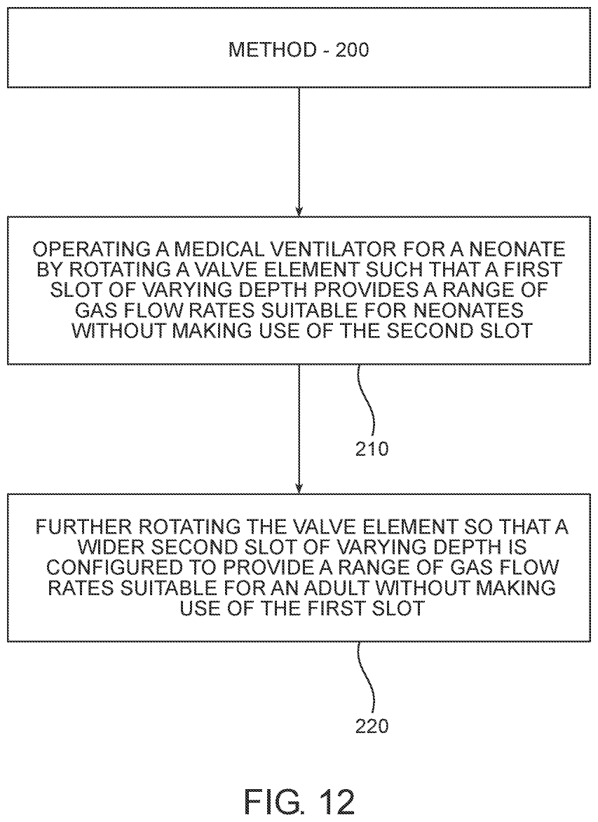

According to some embodiments, as seen in the schematic illustration of FIG. 11, there is further provided a gas control system 100 for ventilating patients, comprising a source of gas 110 such as a blower of a medical ventilator 110, for example of a blower type medical ventilator and a variable orifice rotary valve 10 for controlling gas flow. Valve 10 of system 100 may be any version of valve 10 described in this patent application. System may also comprise at least one flow sensor and at least one pressure sensor 120. The sensor(s) 120 may be situated between (in terms of the flow path) the valve 10 and a patient, P.

Optionally, system 100 may also comprise a processing unit 130 including a processor 131 and programmable instructions or software 132 stored on a computer-readable memory 133, the programmable instructions or software executed by the processor 131. The processor 131 and software 132 are configured to control the valve and/or sensors to determine a suitable rate of flow for the patient based on the patient's age and/or medical condition. System 100 may further comprise an electrical controller (not shown) for controlling the rotational position of the valve element 20 withing the housing of valve 10 by rotating a shaft connected to or within valve element 20. This controls the area of the overlap between a slot in sidewall 22 of valve element 30 and the housing opening 36 and thereby controls the three-dimensional area of any slot that overlaps with housing opening 36. This controls the gas flow rate of the gas flowing through valve 10 of system 100.

Generally speaking, the computer-accessible medium 133 may include any tangible or non-transitory storage media or memory media such as electronic, magnetic, or optical media—e.g., disk or CD/DVD-ROM coupled to computer processing unit 130 (for example via bus) including flash memory. The terms “tangible” and “nontransitory,” as used herein, are intended to describe a computer-readable storage medium (or “memory”) excluding propagating electromagnetic signals but are not intended to otherwise limit the type of physical computer-readable storage device that is encompassed by the phrase computer-readable medium or memory. For instance, the terms “non-transitory computer readable medium” or “tangible memory” are intended to encompass types of storage devices that do not necessarily store information permanently, including for example, random access memory (RAM). Program instructions and data stored on a tangible computer-accessible storage medium in non-transitory form may further be transmitted by transmission media or signals such as electrical, electromagnetic, or digital signals, which may be conveyed via a communication medium such as a network and/or a wireless link.

Another embodiment is a method 200 utilizing a medical ventilator having a housing 30 and a valve element 20 in which the valve element 20 has two slots 26, 28a, as described herein. A first step 210 of the method 200 may comprise rotating the valve element such that a first slot 26 of varying depth is configured to provide a range of gas flow rates suitable for neonates without making use of the second slot. As the valve element 20 is rotated the depth of the first slot (aligned with the housing opening 36) varies and the system 100 is configured to takes into consideration the needs of the particular neonate and the particular breathing patterns occurring. The second step 220 of the method 200 may comprise further rotating the valve element 20 such that a wider second slot 28a of varying depth is configured to provide a range of gas flow rates suitable for an adult without making use of the first slot 26. The two slots 26, 28a may be circumferentially spaced as in FIG. 8a and FIG. 8b.

Other methods using any other version of the valve 10 described herein are also within the invention. For example, another method in which two or more slots are axially separated as in FIG. 8c, FIG. 9, FIG. 10a, FIG. 10b may be used in two separate steps, or another method in which two slots of valve element 20 differ in slope or length or in width etc. may be used in two separate steps of a method.

For example, in another method utilizing a medical ventilator having a housing 30 and a valve element 20 in which the valve element 20 has two slots 26, 28a in accordance with the slots described herein, a first step 210 of the method 200 involves rotating the valve element such that a first slot 26 of varying depth is configured to provide a range of gas flow rates suitable for one adult. As the valve element 20 is rotated the depth of the first slot (aligned with the housing opening 36) varies and the system 100 is configured to takes into consideration the needs of the particular adult and the particular breathing patterns occurring. The second step of this method 200 may comprise further rotating the valve element 20 such that a second slot 28a of varying depth is configured to provide a range of gas flow rates suitable for a different adult. The first slot and the second slot may differ by any of the parameters or versions described herein.

The above are merely a few non-limiting examples or types of methods of using the valve 10 described herein as part of—or not as part of—the system 100 also described herein.

As used herein, the term “about” may be used to specify a value of a quantity or parameter (e.g., the length of an element) to within a continuous range of values in the neighborhood of (and including) a given (stated) value. According to some embodiments, “about” may specify the value of a parameter to be between 90% and 110% of the given value. For example, the statement “the length of the element is equal to about 1 mm” is equivalent to the statement “the length of the element is between 0.9 mm and 1.1 mm.

While the invention has been described with respect to a limited number of embodiments, it will be appreciated that many variations, modifications and other applications of the invention may be made. Therefore, the claimed invention as recited in the claims that follow is not limited to the embodiments described herein.

1.-49. (canceled)

50. A variable orifice rotary valve for controlling gas flow, comprising:
a housing of the valve including a substantially cylindrical interior passage, and a housing opening extending from the interior passage through the housing;
a rotatable valve element comprising a sidewall having a cylindrical external sidewall surface and an internal wall surface, the valve element having a main opening;
at least one slot in the sidewall, the at least one slot varying in depth along a circumferential length of the at least one slot and terminating in an opening in the sidewall,
the valve element rotatably received within the interior passage of the housing such that a rotation of the valve element within the interior passage determines a circumferential length and a depth of a portion of the slot that overlaps with the housing opening.

51. The valve of claim 50, wherein at a position of the valve element in which the at least one slot overlaps with the housing opening, the at least one slot comprises part of a fluid channel from a main opening of the valve element to and through the housing opening, a gas flow rate through the fluid channel determined at least in part by the circumferential length and the depth of the portion of the at least one slot that overlaps with the housing opening.

52. The valve of claim 50, wherein a rate of gas flow varies with a rotational angle of the valve element.

53. The valve of claim 50, wherein the at least one slot is part of a series of one or more slots in the sidewall in which each slot of the series terminates in an opening in the sidewall and differs in regard to one or more of (i) slope, (ii) a width or a change of width, (iii) a depth or a change of depth, (iv) circumferential length, and (v) circumferential length of the opening of the slot.

54. The valve of claim 50, wherein the depth or a rate of change of the depth of the at least one slot or a portion thereof varies substantially along a direction of rotation of the valve element, varies linearly along the circumferential length of the at least one slot, varies nonlinearly along the circumferential length of the at least one slot, varies gradually, and/or varies through a first portion of the at least one slot at a first steepness and then varies through a second portion of the at least one slot at a second steepness.

55. The valve of claim 50, wherein an external wall surface of the housing is cylindrical.

56. The valve of claim 50, wherein the internal wall surface of the rotatable valve element is cylindrical.

57. The valve of claim 50, wherein the at least one slot is a first slot and further comprising a second slot in the sidewall that varies in depth along a circumferential length of the second slot and terminates in a second opening in the sidewall.

58. The valve of claim 57, wherein a rate of change in the depth of the second slot differs from a rate of change of the depth of the first slot.

59. The valve of claim 57, wherein the first slot and the second slot are circumferentially spaced from one another so that the first slot overlaps with the housing opening when the second slot does not overlap with the housing opening and the second slot overlaps with the housing opening when the first slot does not overlap with the housing opening; or
wherein a first part of the first slot is circumferentially spaced from the second slot and a second part of the first slot overlaps circumferentially with the second slot such that when the first part of the first slot overlaps with the housing opening the second slot does not overlap with the housing opening and when the second part of the first slot overlaps with the housing opening the second slot overlaps with the housing opening.

60. The valve of claim 57, wherein the second slot is substantially parallel to the first slot, and wherein the first slot gradually deepens for a first number of rotational degrees, the second slot gradually deepens for a second number of rotational degrees, the second amount beginning at an end of the first slot; or
wherein the second slot is substantially parallel to the first slot, and wherein the first slot gradually deepens for a first number of rotational degrees, the second slot gradually deepens for a second number of rotational degrees, the second number of rotational degrees beginning at the opening of the sidewall of the first slot; or
wherein a second slot is substantially parallel to the first slot, wherein the first slot gradually deepens for a first number of rotational degrees until a first point at which the depth of the first slot remains constant, the second slot gradually deepening for a second number of rotational degrees, the second number of rotational degrees beginning from the first point at which the depth of the first slot remains constant or from a later point during which the first slot remains at a constant depth.

61. The valve of claim 57, wherein the first slot is in a different axial position from the second slot.

62. The valve of claim 57, wherein the first slot and the second slot are not parallel to one another.

63. The valve of claim 57, wherein the first slot is configured to provide a range of gas flow rates suitable for adults and the second slot is configured to provide a range of gas flow rates suitable for neonates.

64. The valve of claim 57, further comprising a third slot in the sidewall that varies in depth along a circumferential length of the third slot and terminates in a third opening in the sidewall.

65. The valve of claim 64, wherein at least two of the first slot, the second slot and the third slot have a substantially same slope, substantially same circumferential length, substantially same rate of change of width, and/or substantially identically shaped profile.

66. The valve of claim 64, wherein each of the first slot, the second slot and the third slot has a different slope, a different circumferential length, a different rate of change of width, and/or a differently shaped profile.

67. The valve of claim 50, wherein the housing opening is a first housing opening and further comprising a second housing opening extending from the interior passage through the housing, wherein:
a circumferential position of the first housing is different than a circumferential position of the second housing, or
an axial position of the first housing is different than an axial position of the second housing.

68. A gas control system for ventilating patients, comprising:
a source of gas;
the variable orifice rotary valve of claim 50; and
at least one flow sensor and at least one pressure sensor; and optionally further comprising a processing unit that includes a processor and programmable instructions or software stored on a tangible or non-transitory computer-readable medium, the programmable instructions or software, when executed by the processor, configured to control the at least one flow sensor and at least one pressure sensor.

69. A method of operating a medical ventilator having a valve in which a valve element comprising slots rotates within a housing, the method comprising:
rotating the valve element such that a first slot is configured to provide a range of gas flow rates suitable for neonates without making use of the second slot and such that as the valve element is rotated the depth of the first slot, aligned with a housing opening, varies; and
further rotating the valve element such that a wider second slot is configured to provide a range of gas flow rates suitable for adult without making use of the first slot,
wherein the first slot and the second slot are circumferentially spaced from one another.
